(19) United States
(12) Patent Application Publication
(10) Pub. No.: US 2006/0104951 A1
(43) Pub. Date: May 18, 2006

(54) Akt and regulation of RA synovial fibroblast apoptosis

(76) Inventors: John D. Mountz, Birmingham, AL (US); Huang-Ge Zhang, Birmingham, AL (US); Hui-Chen Hsu, Birmingham, AL (US)

Correspondence Address: NEEDLE & ROSENBERG, P.C., SUITE 1000, 999 PEACHTREE STREET, ATLANTA, GA, 30309-3915, US

(21) Appl. No.: 10/989,722

(22) Filed: Nov. 16, 2004

(51) Int. Cl. A61K 48/00; A61K 31/366

(52) U.S. Cl. 424/93.2; 514/44; 514/453

(57) ABSTRACT

The administration of an Akt inhibitor in a suitable carrier to a rheumatoid arthritis synovial fibroblast affords a process for inducing rheumatoid arthritis synovial fibroblast apoptosis. The Akt inhibitor is administered either as an active molecule or as a gene sequence expressible within rheumatoid arthritis synovial fibroblast cells. The gene sequence can be encompassed within a gene vector such as an adenovirus. A process for assaying rheumatoid arthritis drug candidates for apoptosis affect includes exposing a culture of rheumatoid arthritis synovial fibroblast cells to a drug candidate and monitoring apoptosis in the culture in the presence of the drug candidate. Apoptosis in the culture is compared to apoptosis induced in a duplicate culture in the presence of a known Akt inhibitor.

### FIELD OF THE INVENTION

The present invention relates to Akt regulation of rheumatoid arthritis synovial fibroblast (RASF) apoptosis and, more particularly, to a screening assay for native Akt activity and a method of regulating RA synovial fibroblast apoptosis.

### BACKGROUND OF THE INVENTION

Synovial hyperplasia is a hallmark of rheumatoid arthritis (Feldmann et al. Annual Review of Immunology, 1996; 14:397-440; Arend et al. 1995; Arth. Rheum. 38:151-60). One of the primary cytokines associated with synovial hyperplasia is tumor necrosis factor-alpha (TNF-α). TNF-α is produced primarily by activated macrophages, and is capable of inducing cell proliferation, activation, and apoptosis (Alvaro-Gracia et al. J. Clin. Invest. 1990; 86:1790-8). In synovial fibroblasts, TNF-α drives proliferation (Kim et al. J. Immunol. 2000; 164:1576-81; Aupperle et al. J. Immunol. 1999; 163:427-33; Kobayashi et al. Arth. Rheum. 1999; 42:519-26) and the production of collagenase and stromylysin, as well as other enzymes, that promote invasion of cartilage and bone.

TNF-α activates nuclear translocation of the transcription factor NF-κB and the TNFR-associated death domain-containing protein (TRADD) (Hsu et al. Cell 1995; 19; 81:495-504). TNFR signaling also has been shown to activate phosphatidylinositol 3-kinase (PI 3-kinase) and protein kinase B (Akt), in several cell types (Beraud et al. Proc. Natl. Acad. Sci. USA 1999; 9; 96:429-34; Ozes et al. Nature 1999; 2; 401:82-5; Pastorino et al. J. Biol. Chem. 1999; 2; 274:19411-6; Reddy et al. J. Immunol. 2000; 164:1355-63; Zhou et al. J. Biol. Chem. 2000; 17; 275:8027-8031; Sanz. EMBO J. 1999; 18:3044-53).

Akt is a serine-threonine protein kinase that is regulated by phosphatidylinositol 3,4,5-triphosphate (PIP3) and has been implicated in signaling of survival in a wide variety of cells, including fibroblastic, epithelial, and neuronal cells (Franke et al. Cell 1997; 1; 88:435-7; Hemmings et al. Science 1997; 275:628-30). Akt was first recognized as an anti-apoptosis factor during analysis of signaling by insulin-like growth factor-1 (IGF-1), which promotes the survival of cerebellar neurons (Dudek et al. Science 1997; 275:661-5). IGF-1 was shown to activate PI 3-kinase-triggered activation of the serine-threonine kinase, Akt. An anti-apoptosis role for Akt also has been identified in NIH 3T3 fibroblasts (Goruppi et al. Mol. Cell Biol. 1997; 17:4442-53).

The phosphatase and tensin homologue deleted on chromosome 10 gene (PTEN) is a phosphatase and tensin homologue suppressor gene located on human chromosome 10q23 (Li et al. Science 1997; 275:1943-7; Steck et al. Nat. Genet. 1997; 15:356-62; Olschwang et al. Nat. Genet. 1998; 18:12-4; Liaw et al. Nat. Genet. 1997; 16:64-7). PTEN is deleted or mutated in a wide range of human cancers, including glioblastoma, melanoma, and prostate, breast, and endometrial cancers. The domains of PTEN share a high degree of homology with the family of protein-tyrosine phosphatases and the cytoskeletal protein, tensin. PTEN functions as a dual-specificity phosphatase and lipid phosphatase in vitro (Furnari et al. Proc. Natl. Acad. Sci. USA 1997; 94:12479-84). Specific substrates include phosphatidylinositol 3,4,5-trisphosphate (PIP3). PTEN has been shown to increase the sensitivity of the cell death response to several apoptotic stimuli, including UV irradiation and treatment with TNF-α.

Previous investigators have reported that ceramides exhibit a growth-promoting anti-apoptotic signal in rheumatoid synovial cells, and that treatment with C2-ceramide completely inhibits PDGF-induced cell cycle progression of rheumatoid synovial cells (Romashkova et al. Nature 1999; 401: 86-89). Increased activity of Akt has been reported in 293 cells and is involved in the activation of NF-κB by TNF-α, following the activation of PI3-kinase. Constitutively active Akt induces NF-κB activity, mediated by phosphorylation of IκBK-α (Ozes et al. Nature 1999; 2; 401:82-5). In contrast, platelet-derived growth factor (PDGF), but not TNF-α, has been reported to activate NF-kB through activation of Akt in normal human skin fibroblasts and in rat fibroblast-like synoviocytes (Romashkova et al. Nature 1999; 401: 86-89).

RASF exhibit increased proliferation in vitro, an increased response to TNF-α and increased production of matrix metalloproteinases (Zhang et al. Cell Dev. Biol. 1997; 33:37-415). It has been shown previously that TNF-α signaling of the TNF receptor results in phosphorylation of IκB and degradation by the proteosome (Ozes et al. Nature 1999; 2; 401:82-5; Zhang et al. Arth. Rheum. 2000 43(5):1094-105). This leads to nuclear translocation of NF-κB which upregulates anti-apoptosis genes including XIAP (Zhang et al. Arth. Rheum. 2000; 43(5):1094-105; Reddy et al. J. Immunol. 2000; 1; 164:1355-6). PI3-kinase has recently been shown to induce NF-κB activation (Reddy et al. J. Immunol. 2000; 1; 164:1355-6) and TNF receptor signaling has been shown to activate Akt (Ozes et al. Nature 1999; 2; 401:82-5; Reddy et al. J. Immunol. 2000; 1; 164:1355-6).

While Akt has an anti-apoptosis role in regard to IGF-1 and NIH 3T3 fibroblasts, no apoptotic role has been determined in regard to RASFs. Determination of Akt operation in RASF apoptosis has implications in suppressing synovial hyperplasia.

### SUMMARY OF THE INVENTION

A process of inducing rheumatoid arthritis synovial fibroblast apoptosis includes the step of administering an Akt inhibitor in a suitable carrier to a rheumatoid arthritis synovial fibroblast. An Akt inhibitor in a suitable carrier in contact with a rheumatoid arthritis synovial fibroblast represents a composition useful in inducing fibroblast apoptosis. A vector such as an adenovirus includes a gene expressible within rheumatoid arthritis synovial fibroblasts, the gene encoding a polypeptide having an inhibitory effect on a Akt.

A process for assaying rheumatoid arthritis drug candidate apoptosis affect includes the steps of exposing a culture of rheumatoid arthritis synovial fibroblast to a drug candidate and monitoring apoptosis of the culture in the presence of the drug candidate. A comparison of apoptosis in the culture to apoptosis induced in a duplicate culture in the presence of a controller known Akt inhibitor affords a measure of drug candidate efficacy.

### DESCRIPTION OF THE DRAWINGS

FIG. 1(A-F) are micrographs of primary RASF (A, C, E) and OASF (B, D, F) grown to confluence and stimulated with either PBS control (A, B) with TNF-α (10 ng/ml) (C, D) or TNF-α (10 ng/ml) plus wortmannin (50 nM) (E, F). After 6 hours, the cells were washed and stained with an anti-phosphorylated Akt (Thr308) antibody and revealed by DAB substrate. Cells were photographed at 40×.

FIG. 2(A) are Western blots of cellular extracts prepared from RASF and OASF treated either with PBS, TNF-α, or TNF-α plus wortmannin as described in FIG. 1. The levels of phosphorylated-Akt and total Akt were determined by Western blot analysis of lysates from RASF and OASF cell lines. Identical blots were probed with an antibody for: upper panel, phosphorylated Akt; middle panel, Akt; and lower panel, anti-b-actin. Lane 1, RASF treated with PBS; lane 2, RASF treated with TNF-α 10 ng/ml); lane 3, RASF treated with TNF-α 10 ng/ml+wortmannin (50 nM); lane 4, OASF treated with PBS; lane 5, OASF treated with TNFα 110 ng/ml); lane 6, OASF treated with TNF-α 10 ng/ml)+wortmannin (50 nM). (B) are Akt-kinase activity in RASF and OASF. Akt from equal amounts of total proteins was immunoprecipitated from RA cell line RA68 or an OA cell line. The Akt kinase activity of cell lysates was revealed using a GSK-3 fusion protein. Lane 1, unstimulated; lane 2, stimulation with TNF-α (10 ng/ml); lane 3, stimulation with TNF-α (10 ng/ml)+wortmannin (50 nM); lane 4, treatment with SV40 DNA which stimulates Akt-kinase activity.

FIG. 3 is a histogram showing apoptosis of RASF cells treated with TNF-α ng/ml or wortmannin alone or TNF-α (10 ng/ml) with different concentrations of wortmannin determined after 18 hours by ATPlite assay. Each data point represents the mean+/−SEM of 5 replications for each bar. (*) indicates groups that are statistically significant different (p<0.05) compared to the other groups.

FIG. 4(A-B) are micrographs showing RASF cells transfected with (A) AdGFP or (B) AdAkt-DN (50 pfu/cell) and cultured for 18 hr. and analyzed for expression of phosphorylated Akt by immunohistochemical staining at 40×. FIG. 4(C) is a Western blot of RASF transfected with either 50 pfu/cell of AdGFP (lane 1), or with AdAkt-DN at a concentration of 5 pfu/cell (lane 2) or 50 pfu/cell (lane 3) for 18 hr. The cells were stimulated with TNFα (10 ng/ml) for 18 hr and Western blot analysis was carried out using an anti-phosphorylated Akt antibody or an anti-β-actin antibody.

FIG. 5(A-D) are a histologic analyses of apoptosis of RASF transfected by AdAkt-DN in the presence of TNF-α. RASF cell lines were transfected with (A) AdGFP (100 pfu/cell) or different amounts of AdAkt-DN and cultured for 18 hr. (B) 5 pfu/cell; (C) 50 pfu/cell; (D) 100 pfu/cell. The cells were then incubated with TNF-α (10 ng/ml) for an additional 18 hr. The apoptosis was determined by microscopic analysis of morphology using an inverted fluorescent microscope.

FIG. 6 is a plot of cytotoxicity of synovial fibroblasts as a function of AdAkt-DN transfection quantity (pfu/cell) in the presence of TNF-α for 3 different RASF and OASF cell lines. Cytotoxicity was quantitated using the ATPLite assay. There was no significant cytotoxicity of either RASF or OASF 18 hr after transfection with AdGFP in the presence of TNF-α (10 ng/ml) (dashed line).

FIG. 7 is a plot of cytotoxicity of synovial fibroblasts as a function of AdPTEN transfection quantity (pfu/cell) in the presence of TNF-α for 3 different RASF and OASF cell lines. Cytotoxicity was quantitated using the ATPLite assay. There was no significant cytotoxicity of either RASF or OASF 18 hr after transfection with AdPTEN followed by incubation with TNF-α (dashed line).

### DETAILED DESCRIPTION OF THE INVENTION

The expression and activation of Akt in synovial fibroblasts from patients with rheumatoid arthritis (RASF), using synovial fibroblasts obtained from patients with osteoarthritis (OASF) as the controls is operative according to the present invention as a heretofore unknown method of inducing apoptosis in RASF.

The present invention derives from the discovery that the levels of phosphorylated-Akt are higher in rheumatoid arthritis synovial fibroblasts (RASF) than in osteoarthritis synovial fibroblasts (OASF), as demonstrated by immunohistochemical staining, immunoblot analysis and an Akt kinase assay. The levels of Akt, phosphorylated Akt and Akt kinase activity are increased by stimulation of primary RASF with tumor necrosis factor-alpha (TNF-α). Treatment of RASF with the PI 3-kinase inhibitor, wortmannin, and a constant dose of TNF-α resulted in apoptosis of greater than half of RASF within 24 hours. This pro-apoptosis effect is specific for Akt, as equivalent levels of apoptosis are observed upon TNF-α treatment of RASF transfected with an adenovirus expressing a dominant negative-Akt (AdAkt-DN). Similarly, apoptosis is induced by TNF-α treatment of RASF transfected with an adenovirus expressing PTEN (AdPTEN), which opposes the action of Akt, indicating that phosphorylated Akt acts as a survival signal in RASF and contributes to the stimulatory effect of TNF-α on these cells by inhibiting the apoptosis response. This effect is not observed in OASF.

In another embodiment, the present invention is operative as a process for assaying RA apoptotic drug candidate efficacy. A drug candidate is exposed to a RASF cell culture prepared as described herein. Apoptosis is monitored in the cell culture as a function of time. It is appreciated that the cell culture is optionally stimulated towards apoptosis with an apoptotic stimulant such as TNF-α or other TNF isoforms. Upon comparison of cell culture apoptosis in the presence of a drug candidate to a control culture exposed to a substance having a known apoptotic effect, the efficacy of the drug candidate is assessed.

It is noted that catalytically active variants and fragments of Akt inhibitors are also operative according to the present invention to induce RASF apoptosis.

The present invention provides for an Akt inhibitor; wherein the Akt inhibitor is a molecule illustratively including a cyclooxygenase-2 inhibitor, a pyridinyl imidazole inhibitor, a Ber-Abl tyrosine kinase inhibitor and a PI-3 kinase inhibitor. An example of a pyridinyl imidazole is SB203580 commercially available from Calbiochem-Novabiochem. An example of a Ber-Abl tyrosine kinase inhibitor is CGP57148B, also known as STI-571, made by Novartis Pharma AG. An example of a PI-3 kinase inhibitor is LY294002, also known as 2-(4-morpholinyl)-8-phenyl-4H-1-benzopyran-4-one, commercially available from Calbiochem. An example of a cyclooxygenase-2 inhibitor is celecoxib.

It will be appreciated by those skilled in the art that Akt inhibition is achieved by inhibition of factors that cause an increase in Akt levels, activity or phosphorylation or which are necessary for Akt activation. Factors known to increase Akt or which are necessary for Akt activation illustratively include insulin-like growth factor-1, IL-1, PDGF, focal adhesion kinase, lipoarabinomannan and Syk.

It will be appreciated by those skilled in the art that stimulation of Akt activation is useful in inhibiting apoptosis.

The present invention provides for an Akt inhibitor protein or expressible gene to an RASF through incubation exposure or gene delivery, respectively; wherein the Akt inhibitor polypeptide includes a PI-3 kinase inhibitor such as wortmannin, and the Akt inhibitor expressing gene encodes a phosphatase such as PTEN, or dominant-negative Akt. Certain truncations of these proteins or genes perform the regulatory or enzymatic functions of the full sequence protein or gene. For example, the nucleic acid sequences coding therefor can be altered by substitutions, additions, deletions or multimeric expression that provide for functionally equivalent proteins or genes. Due to the degeneracy of nucleic acid coding sequences, other sequences which encode substantially the same amino acid sequences as those of the naturally occurring proteins may be used in the practice of the present invention. These include, but are not limited to, nucleic acid sequences including all or portions of the nucleic acid sequences encoding the above polypeptides, which are altered by the substitution of different codons that encode a functionally equivalent amino acid residue within the sequence, thus producing a silent change. For example, one or more amino acid residues within a polypeptide sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Also included within the scope of the present invention are proteins or fragments or derivatives thereof which are differentially modified during or after translation, e.g., by glycosolation, protolytic cleavage, linkage to an antibody molecule or other cellular ligands, etc. in addition, the recombinant vector encoding nucleic acid sequences of the present invention Akt inhibitor may be engineered so as to modify processing or expression of a vector. For example, a signal sequence may be inserted upstream of an inhibitor encoding sequence to permit inhibitor secretion and thereby facilitate apoptosis.

Additionally, an inhibitor encoding nucleic acid sequence can be mutated in vitro or in vivo to create and/or destroy translation, initiation, and/or termination sequences or to create variations in coding regions and/or form new restriction endonuclease sites or destroy pre-existing ones, to facilitate further in vitro modification. Any technique for mutagenesis known in the art can be used, including but not limited to in vitro site directed mutagenesis, J. Biol. Chem. 253:6551, use of Tab linkers (Pharmacia), and the like.

The present invention further provides for an Akt inhibitor that is an antisense Akt nucleic acid. In order to inhibit Akt by an antisense approach, oligonucleotides (either DNA or RNA) that are complementary to Akt mRNA are synthesized and administered by methods known to those skilled in the art. In addition, an anti-Akt antibody is appreciated to act as an AKT inhibitor. Such an antibody is created by methods common to the art.

An Akt inhibitor polypeptide or gene vector and TNF-α are administered in vivo by modes illustratively including parenterally, intrasynovially and topically with a carrier or diluent suitable for the mode of administration. Suitable carriers and diluents for each mode of administration are known to those skilled in the art. It is appreciated that additional adjuvants known to those skilled in the art are optionally added to a carrier or diluent. Thus, an Akt inhibitor formulation suitable for injection illustratively includes aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives.

The present invention operates to inhibit expression of activated Akt that plays a central role in inhibition of apoptosis of RASF. Thus, apoptosis resistance enables cells that normally occur at a low frequency to become the predominant cell type within a pathologic lesion. The disablement of the resistance mechanism is expected to return normal cell type proportions. The enhanced expression of phosphorylated Akt in early-passage primary RASF is noted as an indicator for utilization of the present invention.

The present invention demonstrates that Akt is constitutively activated, and further activated in response to TNF-α, in early passage synovial fibroblasts from several patients with RA and that inhibition of Akt serves to induce RASF apoptosis. Lead molecules that affect Akt activity are summarized in Table 1.

TABLE 1
Lead Molecules that Affect Akt

| | Action | Cell | Reference |
|---|---|---|---|
| Small Compound | | | |
| cyclo-oxygenase 2 | Block Akt | Prostate cancer | Hsu et al. |
| inhibitor | | | JBC 275, 11397, |
| (Celebrex) | | | 2000 |
| Celecoxib | | | |
| Pyridinyl | Block Akt | IL-2 activated | Lali et al. |
| imidazole inhibitor | | T cell | JBC 275, 7395, |
| SB 203580 | | | 2000 |
| Bcr-Abl specific | Block Akt | HL-60 K562 | Fang et al. |
| tyrosine kinase | (decrease IAP, | | Blood 96, 2246, |
| inhibitor | Bcl-xL NFκB) | | 2000 |
| CGP57148B (STI- | | | |
| 571) | | | |
| Biologics | | | |
| Compound | | | |
| lipo | Activates Akt | THP = 1 cell | Maiti et al. |
| arabinomannin | | | I. Biol. Chem. |
| (LAM) for | | | October 2000 |
| M. Tuberculosis | | | |
| Pathways | | | |
| Compound | | | |
| Insulin-like growth | Activates Akt | Many | Many |
| factor-1, IL-1, | | | |
| PDGF | | | |
| SSby | Activates Akt | H₂O₂ treated B | Ding et al. |
| | | cells | JBC 275, 30873, |
| | | | 2000 |
| Focal adhesion | Activates Akt | HL-60 | Sohoda et al. |
| kinase | | | JBC 275, 16309, |
| | | | 2000 |

Akt activation is activated by TNF-α ligation of the TNFR, and this leads to increased NF-κB activation in RASF. It will be appreciated that Akt is activated by multiple members of the TNFR superfamily known to those skilled in the art, illustratively including TNFR-I, TNFR-II and LT-beta R. It will be further appreciated by those skilled in the art that multiple members of the class of TNFR superfamily ligands, illustratively including TNF-α, TNF-β, TRAIL, LT-beta and heteromers of these or other ligands bind to TNFR superfamily members to activate Akt. In addition, TNFR-activating antibodies, such as SSR539 commercially available from R and D Systems, activate Akt.

Akt activity is found to be constitutively higher in RASF compared to OASF. This is shown by analysis of phosphorylated Akt from RASF and OASF, and also by immunoprecipitation of activated Akt and evaluation of activity using GSK-3 as the substrate. Furthermore, Akt activity is greatly enhanced in RASF in the presence of TNF-α, but this enhancement was not observed in OASF. Thus, there is an intrinsic dysfunction associated with the higher expression of activated Akt in RASF than in OASF as a result of genetic or environmental factors, leading to enhanced activation of Akt in response to TNF-α. Over expression of TNF-α combined with this intrinsic enhanced expression of activated Akt leads to inhibition of apoptosis of RASF and, consequently, a growth advantage for these synovial fibroblasts. Since rheumatoid arthritis is a chronic inflammatory disease that is characterized by a synovial hyperplasia that develops over many years, there is gradual selection for RASF that express high levels of phosphorylated Akt in response to TNF-α. This is consistent with the inventive intervention demonstrating that 45% of RASF constitutively express phosphorylated Akt, and undergo TNF-α mediated apoptosis in response to blocking phosphorylation of Akt with Akt-DN or AdPTEN. In contrast, OASF have not developed an anti-apoptosis pathway that depends upon phosphorylated Akt, and subsequently, blocking of phosphorylated Akt activity does not result in increased susceptibility to apoptosis in response to TNF-α

Akt is regulated in RASF by addition of TNF-α according to the present invention. TNF-α is produced at high levels by the macrophages present in rheumatoid synovium and inhibition of TNF-α by soluble TNF-α receptors is one of the most efficacious therapies for rheumatoid arthritis (sTNFRII, Etanercept) (Moreland et al. N. Engl. J. Med. 1997, 337:141-7; Weinblatt et al. N. Engl. J. Med. 1999, 340:253-9) and sTNFRI (McCabe et al. Arth. Rheum. 1998, 9(supplement) 558; Edwards C K 3rd. Ann. Rheum. Dis. 1999, 58 Suppl 1:I73-81; Bendele et al. Clin. Exp. Rheumatol. 1999, 17:553-60; Su et al. Arthritis Rheum. 1998, 41:139-49). The present results suggest that one of the mechanisms contributing to the efficacy of anti-TNF-α therapy in RA is the down-modulation of the activation of Akt thereby indicating a novel mode of inducing RASF apoptosis and alleviating RA symptoms.

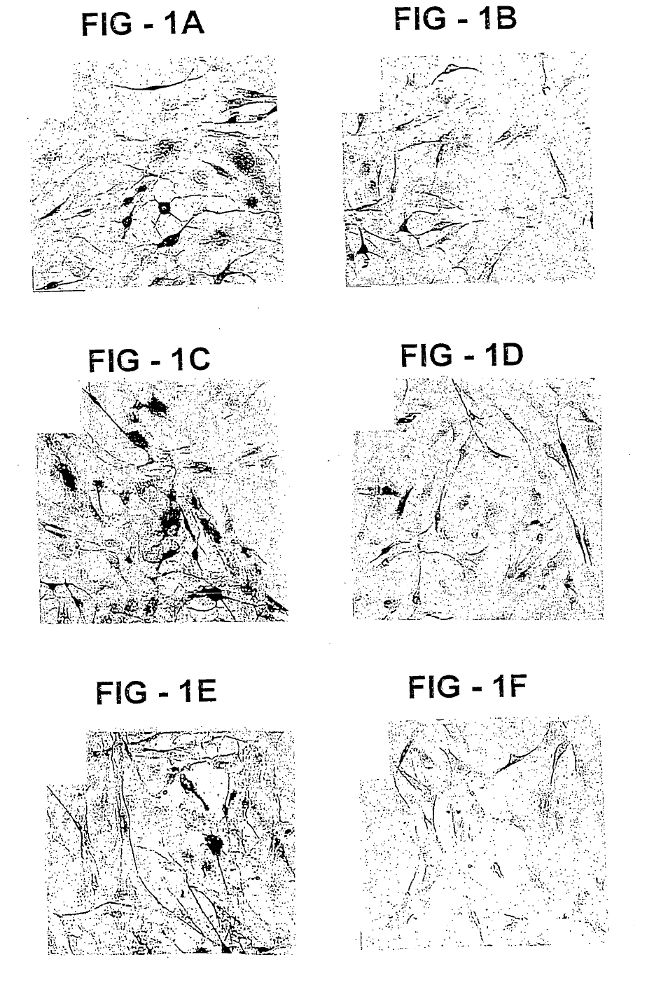

It has been shown that treatment with TNF-α results in increased proliferation of RASF but not OASF (Migita et al. Biochem. Biophys. Res. Commun. 2000; 269:70-5). To determine if this TNF-α survival signal involves a PI 3-kinase dependent mechanism, RASF and OASF are treated with TNF-α (10 ng/ml), using PBS as a control, in the presence and absence of the PI 3-kinase inhibitor, wortmannin (50 nM). After 18 hours, the synovial fibroblasts are stained using an antibody specific for phosphorylated-Akt. The constitutive expression of phosphorylated Akt is higher in the PBS-treated RASF than in the OASF as shown in FIGS. 1A and 1B. Treatment with TNF-α resulted in a greatly enhanced production of phosphorylated Akt by RASF, whereas this effect was much less pronounced in OASF as shown in FIGS. 1C and 1D). On immunohistochemical staining, strong expression of phosphorylated-Akt is detected in 45±10% of primary RASF compared to 20±8% of primary OASF. Treatment with the PI 3-kinase inhibitor, wortmannin inhibited this increase in expression of phosphorylated Akt in response to TNF-α in RASF as shown in FIGS. 1E and 1F. Thus, primary RASP cells exhibit higher expression of phosphorylated Akt than do OASF cells in the absence of exogenous stimulation with TNF-α. Moreover, stimulation with TNF-α greatly increased the expression of phosphorylated Akt in RASF and this enhanced expression requires PI 3-kinase activation.

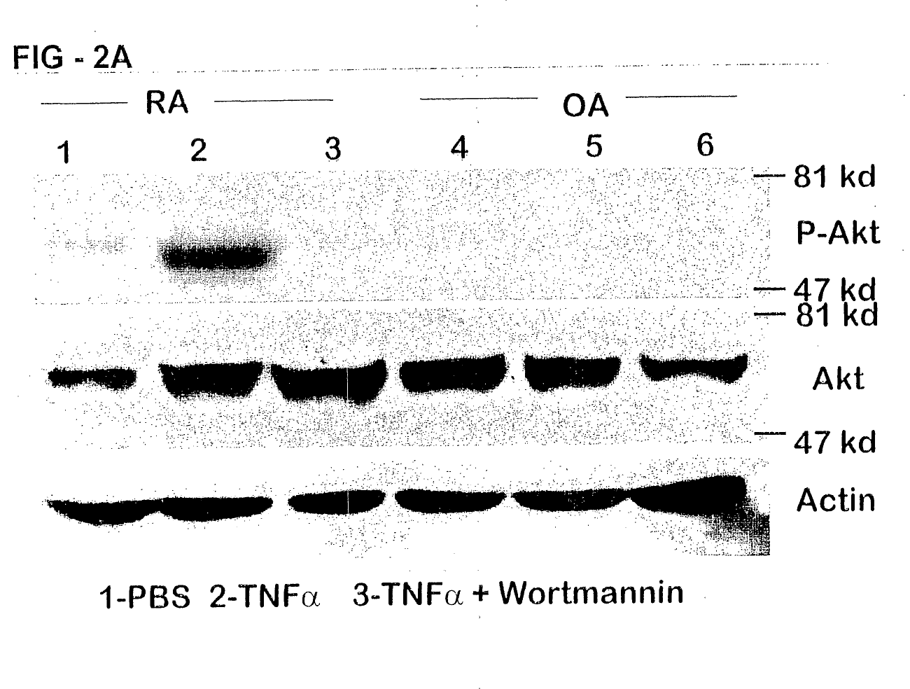

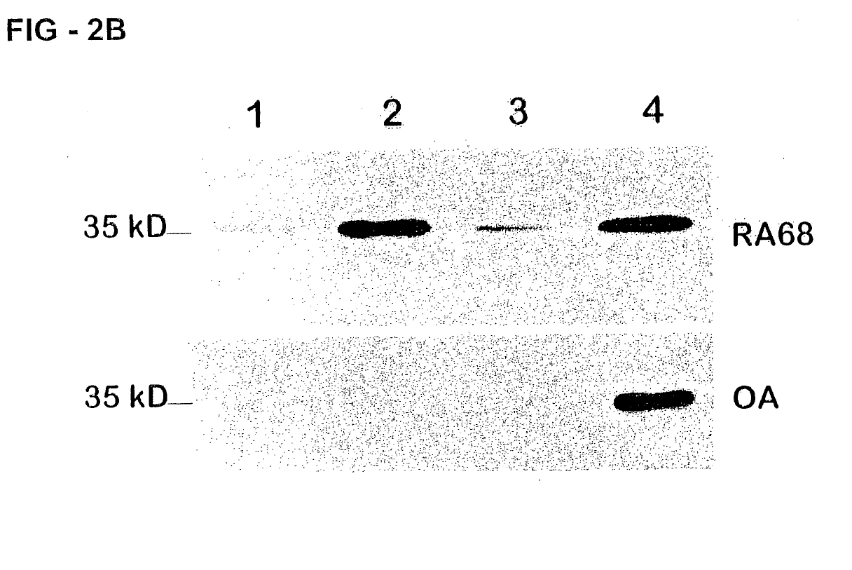

To confirm the results of the histologic analyses, cellular extracts are prepared from RASF and OASF treated either with PBS, TNF-α, or TNF-α plus wortmannin. The levels of phosphorylated-Akt are determined by Western blot analysis of lysates from RASF and OASF cell lines. The levels of phosphorylated Akt are higher in the unstimulated RASF compared to the OASF as shown in FIG. 2A, top panel, lane 1,4. TNF-α results in a marked increase in the levels of phosphorylated-Akt in RASF, but did not affect the levels of phosphorylated Akt in OASF as shown in FIG. 2A, top panel, lane 2,5. This increase in phosphorylated Akt in RASF is inhibited by wortmannin, as shown in FIG. 2A, top panel, lane 3. There is no significant change in total Akt in RASF as compared to OASF independent of treatment, as shown in FIG. 2A, middle panel. Regardless of treatment, the level of β-actin protein is not changed, which indicates that equal amounts of total protein have been loaded as shown in FIG. 2A, lower panel.

To directly determine the Akt-kinase activity in RASF and OASF, Akt from equal amounts of total protein is immunoprecipitated from cell lysates and the kinase activity of Akt revealed using a GSK-3 fusion protein as the substrate in the presence of ATP and kinase buffer. Active Akt phosphorylates GSK-3 at serine 219. The Akt-kinase activity is relatively low in unstimulated RASF line RA68 as shown in FIG. 2B, lane 1, top panel. However, 24 hours after stimulation of RASP with TNF-α (10 ng/ml), there is a 20-fold increase in Akt-kinase activity as shown in FIG. 2B, lane 2, top panel, and this increase in activity is blocked by pretreatment with wortmannin as shown in FIG. 2B, lane 3, top panel. In contrast, untreated OASF exhibited no detectable Akt kinase activity and treatment with TNF-α (10 ng/ml) did not induce Akt kinase activity in OASF as shown in FIG. 2B, lower panel. Cells transfected with an SV40 expression plasmid, which strongly stimulates Akt-kinase activity in cells (Summers et al. Biochem. Biophys. Res. Commun. 1998; 246:76-81) were used as a positive control as shown in FIG. 2B, lane 4, top and lower panels.

Thus, the intrinsic activation of Akt is higher in RASF than OASF. Furthermore, stimulation with TNF-α greatly enhances Akt activity in RASF, but has no effect on Akt activity in OASF. These results support the histologic evidence that the levels of phosphorylated Akt are higher in RASF than in OASF as shown in FIG. 1.

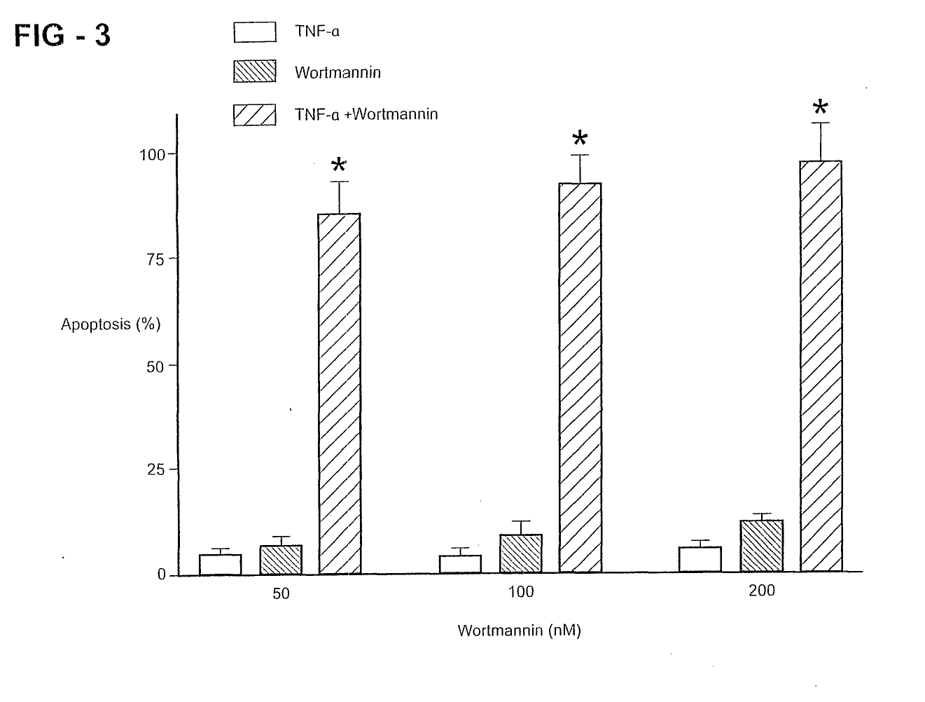

RASF cells are treated with TNF-α alone or TNF-α with wortmannin (50 nM), and the extent of apoptosis monitored by the ATPlite assay. Neither treatment with TNF-α nor wortmannin alone could induce cytotoxicity in RASF as shown in FIG. 3, whereas treatment with TNF-α plus wortmannin resulted in extensive apoptosis. This is consistent with the ability of TNF-α to activate PI 3-kinase in these cells and indicates that TNF-α mediated apoptosis of RASF is potentiated by inhibition of PI 3-kinase activity.

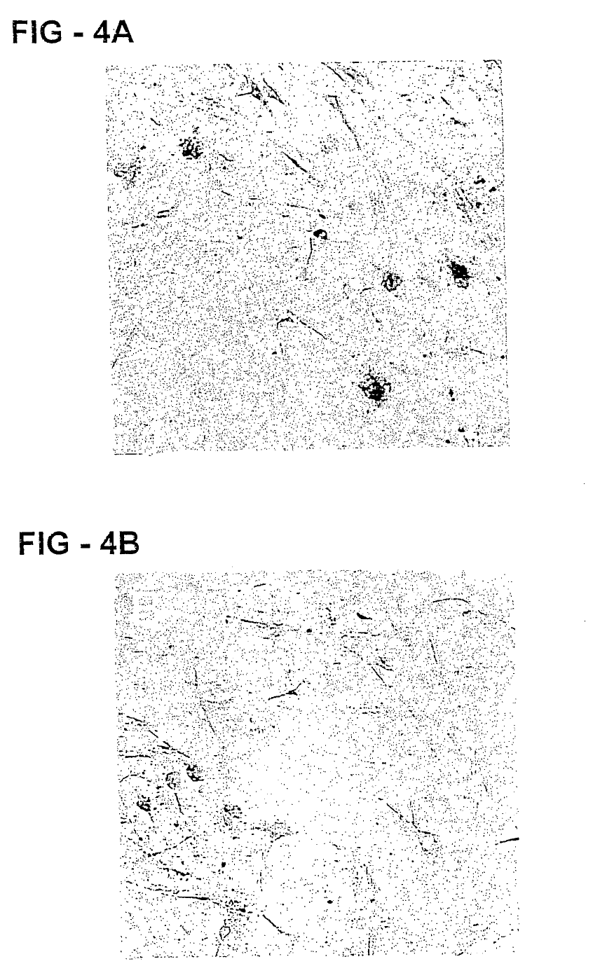

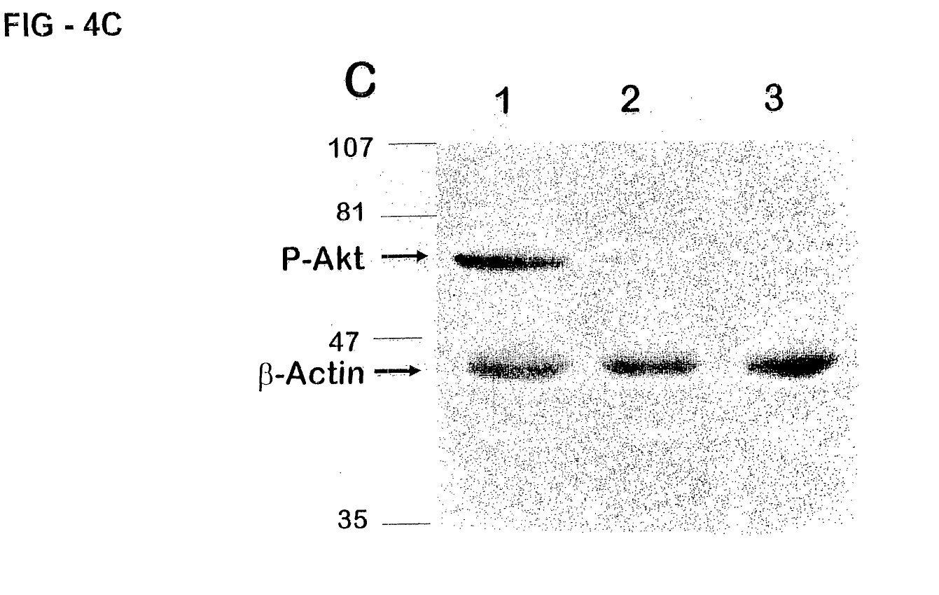

The expression of phosphorylated Akt is higher in the primary cultures of cells from patients with RA than in the primary cultures of cell from patients with OA as shown in FIG. 1. AdAkt-DN inhibits endogenous phosphorylation of Akt. A control, AdGFP shown in FIG. 4A or AdAkt-DN shown in FIG. 4B is transfected (50 pfu/cell) into an RASF cell line. The cells are cultured 18 hr, and analyzed for expression of phosphorylated Akt by immunohistochemical staining. Decreased expression of phosphorylated Akt by RASF transfected with AdAkt-DN is noted compared to RASF transfected with AdGFP.

To determine the optimal amount of AdAkt-DN required to inhibit Akt phosphorylation, RASF is transfected with either 50 pfu/cell of AdGFP or with 5 pfu/cell or with 50 pfu/cell of AdAkt-DN respectively for 18 hr. The cells are then stimulated with TNFα (10 ng/ml) for 18 hr. Phosphorylated Akt in RASF increased 18 hours after stimulation with TNF-α (10 ng/ml) in cells transfected with AdGFP as shown in FIG. 4C, Lane 1. AdAkt-DN inhibits TNFα induced phosphorylation of Akt in a dose-dependent fashion as shown in FIG. 4C, Lane 2 and 3. The total proteins are loaded in equal amounts as indicated by the levels of β-actin as shown in FIG. 4C.

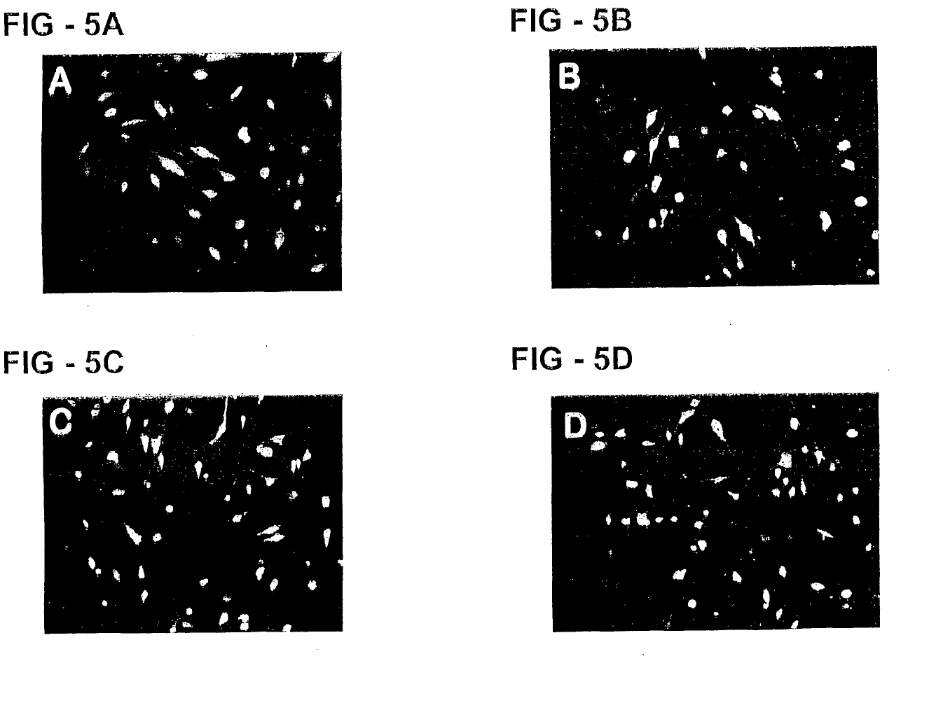

RASF cell lines are transfected with AdGFP (100 pfu/cell) or different amounts of AdAkt-DN (5, 50, 100 pfu/cell) and cultured for 18 hr. The cells are then incubated with TNF-α (10 ng/ml) for an additional 18 hr. Apoptosis is determined by microscopic analysis of the morphology of the RASF using an inverted microscope. There is no apoptosis of RASF after treatment with control AdGFP plus TNF-α as shown in FIG. 5A. There was a dose-dependent increase in apoptosis of RASF after treatment with AdAkt-DN at 5, 50 and 100 pfu/cell, respectively as shown in FIG. 5, B, C, D. Low apoptosis of RASF after transfection with AdAkt-DN is observed in the absence of TNF-α. Thus, treatment with AdAkt-DN plus TNF-α converts the TNF-α apoptosis resistant RASF to TNF-α sensitive RASF.

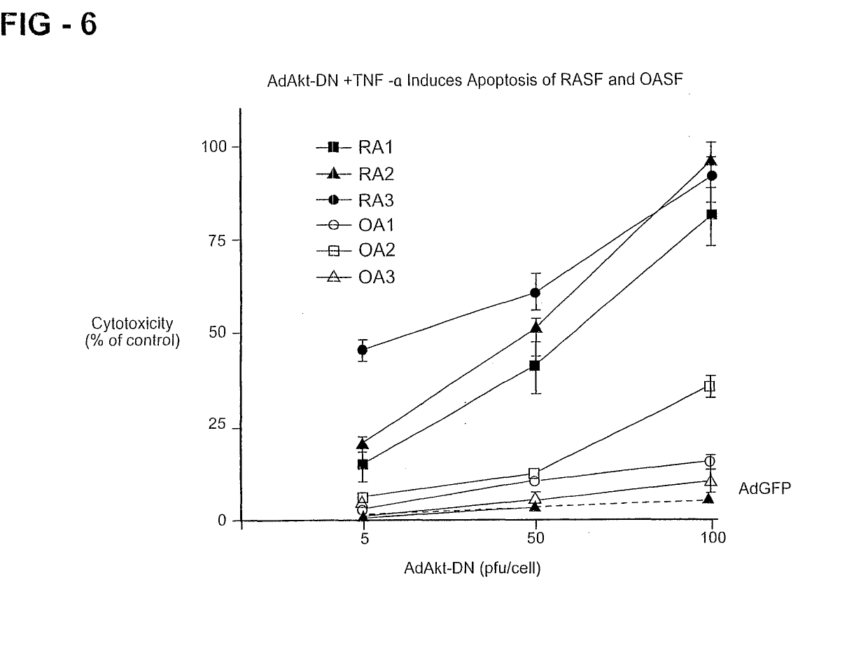

The effect of AdAkt-DN on apoptosis is analyzed using three different primary RASF and primary OASF cell cultures at different pfu/cell of AdAkt-DN using the ATPLite assay. Cells are transfected with AdAkt-DN or AdGFP for 18 hr, and then incubated with TNF-α (10 ng/ml) for an additional 18 hr. There is a dose-dependent increase in cytotoxicity of RASF with increasing pfu/cell of AdAkt-DN in the presence of TNF-α (10 ng/ml) as shown in FIG. 6. In contrast, transfection of OASF with increasing concentrations of AdAkt-DN resulted in only minimal apoptosis after treatment with TNF-α. Neither RASF nor OASF cell lines exhibited significant cytotoxicity after transfection with AdGFP in the presence of TNF-α (10 ng/ml) as demonstrated for exemplary RASF cell line RA2, by the dashed line.

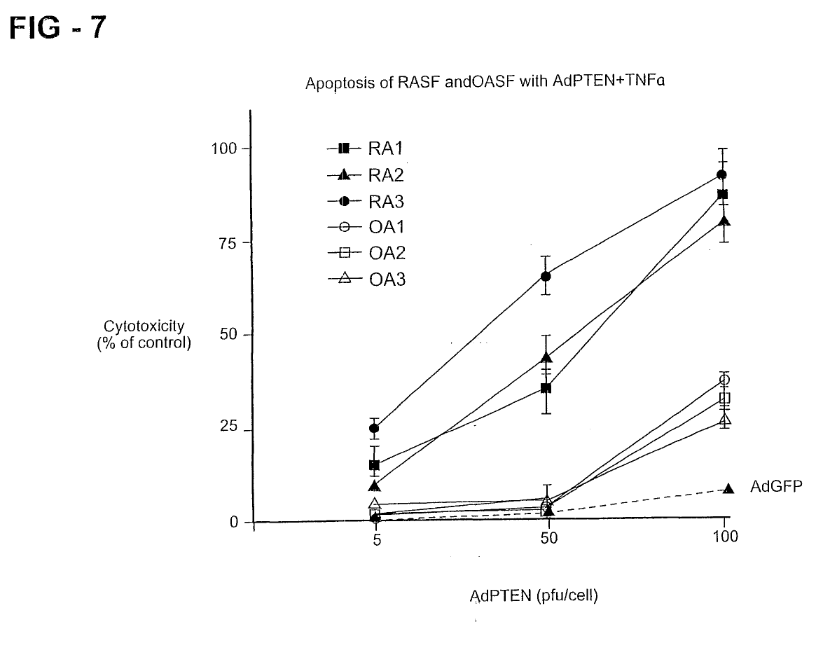

Three different RASF and OASF primary cell cultures are transfected with AdPTEN for 18 hr followed by treatment with TNF-α (10 ng/ml) for 18 hr. Cytotoxicity is analyzed using the ATPLite assay. As a control, cells are treated with AdGFP plus TNF-α as described with respect to FIG. 6. There is a dose-dependent increase in cytotoxicity of RASF with increasing pfu/cell of AdPTEN in the presence of TNF-α (10 ng/ml) as shown in FIG. 7) In contrast, transfection of OASF with increasing concentrations of AdPTEN resulted in only minimal apoptosis after treatment with TNF-α. Neither RASF nor OASF cell lines exhibited significant cytotoxicity after transfection with AdGFP in the presence of TNF-α (10 ng/ml) as demonstrated for an exemplary RASF cell line by the dashed line.

The following examples are given for the purpose of illustrating various embodiments of the invention and are not meant to limit the present invention in any fashion.

### EXAMPLE 1

### Patient Selection

Patients were recruited who were undergoing total knee replacement related to rheumatoid arthritis or osteoarthritis. All patients met the 1987 ACR Criteria for rheumatoid arthritis of the knee (Arnett et al. Arthritis Rheum. 1988; 31:315-24). The diagnosis of OA was based on clinical and radiological findings. The patients ranged in age from 43-77 with a mean age of 63. Synovial cell lines were established from 4 female and 1 male patients. All patients were receiving non-steroidal anti-inflammatory drugs (NSAIDs), but none were receiving steroids or prednisone at the time of surgery.

### EXAMPLE 2

### Reagents

TNF-α was purchased from R&D Systems (Minneapolis, Minn.) and used at 10 ng/ml. This is optimal based on previous data (Zhang et al. Cell Dev. Biol. 1997; 33:37-41). Wortmannin was purchased from Sigma (St. Louis, Mo.) and dissolved in DMSO, aliquoted, stored at −80° C. until use. The SV40 plasmid which expresses the polyoma T antigens was purchased from New England Biolab (Beverly, Mass.) and was used as a positive control for the Akt-kinase assay (Summers et al. Biochem. Biophys. Res. Commun. 1998; 246:76-81).

### EXAMPLE 3

### Primary Synovial Cell Culture

Primary synovial cell lines are established as described previously (Zhang et al. Cell Dev. Biol. 1997; 33:37-41). The synovial tissue is minced into small pieces (˜1 mm³) in prewarmed DMEM then incubated for 2 hours at 37° C. in the presence of 1 mg/ml of type I collagenase (Sigma, St. Louis, Mo.). After dissociation of the fibroblasts, the cells are harvested by centrifugation at 1,000 rpm for 5 min and plated in 25 cm² flasks in 8 ml of DMEM supplemented with 10% fetal bovine serum (FBS). The cells are cultured to 80% confluency before use in experiments. All in vitro experiments are carried out using primary synovial cell cultures at passage numbers between 4-10.

### Example 4

### Construction of AdAkt-DN and an AdPTEN Expression Vector

Akt constructs encoding catalytically inactive Akt (K179M) inserted into pCMV6 vectors and pCMV6 vectors are utilized as previously described (Gu et al. Proc. Natl. Acad. Sci. USA 1997; 94:11345-50). The Akt (K179M) is cloned into the E1A deletion site of adenovirus of pAdCMV (He et al. Proc. Natl. Acad. Sci. USA 1998; 95:2509-2514) in which green fluorescence protein (GFP) is co-expressed with the mutated Akt driven by the CMV promoter, respectively. A recombinant adenovirus is produced by co-transfection of pAdCMVAkt-DN with pJM17 in the 293 cell line. The recombinant adenovirus expressing mutant Akt (AdAkt-DN) is selected and purified using standard procedures (McGrory et al. Virology 1988; 163:614-7). The correct orientation and cloning of the Akt-DN in recombinant AdAkt-DN is confirmed by PCR sequence analysis. To obtain a large quantity of recombinant AdAkt-DN, the 293 cells are infected and grown for 48 hours at 37° C. prior to harvest and centrifugation using a tabletop centrifuge at 4,000 rpm for 20 min. The infected cells are resuspended in PBS buffer, then lysed using three freeze-thaw cycles. The released virus is purified through two CsCI gradients and then the purified recombinant AdAkt-DN is titrated by plaque assay (McGrory et al. Virology 1988; 163:614-7), aliquoted, and stored at −80° C. until use.

The PTEN fragment is constructed as described previously (Ghosh et al. Gene 1999; 235:85-91). The fragment is subsequently cloned into the E1 deleted site of an adenovirus shuttle pAdCMV in which the PTEN gene is driven by the CMV promoter. A recombinant adenovirus is then constructed and characterized as described (Ghosh et al. Gene 1999; 235:85-91).

### EXAMPLE 5

### Immunohistochemical Analysis of Primary Synovial Cell Cultures

The in-situ expression of phosphorylated-Akt is determined by staining fixed cells with an antibody specific for Thr308 phosphorylated Akt (New England Biolabs, Beverly, Mass.). Quenching of endogenous peroxidase is performed by incubating tissue sections with 3% H₂O₂ at RT for 10 min in a humidified chamber. After washing with PBS, tissue sections are incubated with 0.1% trypsin at 37° C. for 10 min to reveal fixed antigen epitopes. Tissue sections are then treated with denaturing solution at 20° C. for 30 min and blocking solution at RT for 10 min followed by incubation with a horseradish peroxidase (HRP) conjugated antibody specific for phosphorylated-Akt antibody (New England Biolabs, Beverly, Mass.). Slides are incubated with DAB staining kit (DAKO) for color development then counterstained with methyl green. At least 5 areas from each specimen are chosen randomly for assessment of the percentage of Akt-positive cells.

### EXAMPLE 6

### Evaluation of Akt Activation and Immunoblot Analysis

For evaluation of the phosphorylated form of Akt, the synovial cells are washed twice with ice-cold PBS followed by incubation with 200 μl of cell lysis buffer (20 mmol/L Tris [pH 7.4], 150 mmol/L NaCl, 1 mmol/L EDTA, 1 mmol/L EGTA, 1% Triton, 2.5 mmol/L sodium pyrophosphate, 1 mmol/L β-glycerophosphate, 1 mmol/L Na₃VO₄, 1 μg/mL leupeptin, and 1 mmol/L PMSF) for 5 min on ice. The cells are then harvested by scraping and sonicated using a Branson Sonicator (3×5 seconds, output control 2, duty cycle 100%) on ice. After centrifugation for 10 min at 20,000 g at 4° C., the protein concentration of the supernatant is determined using the Bio-Rad protein assay kit (Bio-Rad, Hercules, Calif.). Fifty micrograms of total protein extracted from synovial fibroblasts are loaded on 10% SDS-PAGE mini gel (Bio-Rad, Hercules, Calif.), and electrophoresed at 100 V for 2 hours, followed by electro-transfer to nitrocellulose membrane. The membrane is then blotted with an antibody specific for Akt phosphorylated at Ser473 (New England Biolabs, Beverly, Mass.) overnight at 4° C. Phosphorylated Akt is detected using the PhosphoPlus Akt antibody kit (New England Biolabs, Beverly, Mass.). To monitor the amount of total proteins loaded on the gel, the membrane is restripped, and probed with mouse anti-human β-actin antibody (clone AC-15, Sigma, St., Louis, Mo.), and the signal is amplified using HRP-conjugated goat anti-mouse antibody, and detected by the LumiGLO chemiluminescent reagent.

### EXAMPLE 7

### Analysis of AKT Activity

Akt kinase assay of RA synovial fibroblasts is performed using Akt kinase assay kit according to the protocol provided by manufacturer (New England Biolabs, Beverly, Mass.). Briefly, three hundreds micrograms of total proteins from RA synovial fibroblasts are added into Akt antibody coated beads, incubated at 4° C. for 3 hours followed by washing. Phosphorylation of GSK-3 is used as an indicator of phosphorylated Akt, since Akt negatively regulates GSK-3a/b kinase activity via phosphorylation of GSK-3 at Ser219. After the kinase reaction, the reaction mixture is electrophoresed on a 12% SDS-PAGE gel and western blotted. The blots are probed with an anti-phospho-GSK-3a/b (Ser219) antibody (rabbit polyclonal IgG, affinity purified). The blot is developed using an HRP-conjugated goat anti-rabbit antibody, and detected by the LumiGLO chemiluminescent reagent.

### EXAMPLE 8

### ATPlite-M Assay to Analysis Synovial Fibroblast Cytotoxicity

Cytotoxicity is determined by the ATPLite-M assay. ATP is a marker for cell viability because it is present in all metabolically active cells and the concentration declines very rapidly when the cells undergo apoptosis. The ATPLite-M assay system is based on the production of light caused by the reaction of ATP with added luciferase and D-luciferin (Crouch et al. J. Immunol. Methods 1993; 160, 81-88). Briefly, RASF and OASF are transfected with different amounts of control Ad, AdAkt-DN or AdPTEN. Eighteen hr later, the cells are stimulated in a 96 well plate in the presence or absence of TNF-α (10 ng/ml). The cytotoxicity is analyzed by the ATPlite-M assay as described in manual (Packard Instrument Company, Meriden, Conn.).

### EXAMPLE 9

### Statistical Analysis

Data are expressed as mean±SEM from at least 3 independent experiments. Statistical analysis was performed with ANOVA followed by a modified least significant difference test (SPSS Software).

Patent applications and publications mentioned in the specification are indicative of the levels of those skilled in the art to which the invention pertains. These applications and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

### REFERENCES

Hsu et al., The Cyclooxygenase-2 Inhibitor Celecoxib Induces Apoptosis by Blocking Akt Activation in Human Prostate Cancer Cells Independently of Bcl-2, The Journal of Biological Chemistry, 275: 11397, 2000.

Lali et al., The Pyridinyl Imidazole Inhibitor SB203580 Blocks Phosphoinositide-dependent Protein Kinase Activity, Protein Kinase B Phosphorylation, and Retinoblastoma Hyperphosphorylation in Interleukin-2-stimulated T Cells Independently of p38 Mitogen-activated Protein Kinase, The Journal of Biological Chemistry, 275: 7395, 2000.

Fang et al., Blood, 96: 2246, 2000.

Maiti et al., Lipoarabinomannan from Mycobacterium tuberculosis Promotes Macrophage Survival by Phosphorylating Bad through a Phosphatidylinositol 3-Kinase/Akt Pathway The Journal of Biological Chemistry 2000 276: 329-333.

Ding et al., Syk Is Required for the Activation of Akt Survival Pathway in B Cells Exposed to Oxidative Stress, The Journal of Biological Chemistry, 275: 30873, 2000.

Sohoda et al., Anti-apoptotic Role of Focal Adhesion Kinase (FAK). INDUCTION OF INHIBITOR-OF-APOPTOSIS PROTEINS AND APOPTOSIS SUPPRESSION BY THE OVEREXPRESSION OF FAK IN A HUMAN LEUKEMIC CELL LINE, HL-60, The Journal of Biological Chemistry, 275: 16309, 2000.

1. A process of inducing rheumatoid arthritis synovial fibroblast apoptosis comprising the steps of: administering an Akt inhibitor in a suitable carrier to a rheumatoid arthritis synovial fibroblast.

2. The process of claim 1 further comprising administering tumor necrosis factor to said rheumatoid arthritis synovial fibroblast.

3. The process of claim 1 wherein Akt inhibitor and carrier are administered intrasynovially.

4. The process of claim 1 wherein Akt inhibitor and carrier are administered parenterally.

5. The process of claim 1 wherein Akt inhibitor and carrier are administered topically.

6. The process of claim 1 wherein said Akt inhibitor is selected from the group consisting wortmannin, a fragment thereof, and a polymorph thereof.

7. The process of claim 1 wherein said Akt inhibitor is administered as a gene sequence expressible within rheumatoid arthritis synovial fibroblast.

8. The process of claim 7 wherein said gene sequence is encompassed within a gene vector.

9. The process of claim 8 wherein said gene vector is an adenovirus.

10. A composition comprising: an Akt inhibitor in a suitable carrier in contact with rheumatoid arthritis synovial fibroblast.

11. The composition of claim 10 further comprising tumor necrosis factor in said carrier.

12. The composition of claim 10 wherein said Akt inhibitor is a PI 3-kinase inhibitor.

13. The composition of claim 10 wherein said Akt inhibitor is wortmannin.

14. The composition of claim 10 wherein said Akt inhibitor is a vector comprising a gene sequence expressible within the rheumatoid arthritis synovial fibroblast encoding a polypeptide selected from the group consisting of: wortmannin, anti-Akt and dominant negative Akt.

15. The composition of claim 14 wherein said vector is an adenovirus vector.

16. A vector comprising: a gene expressible within rheumatoid arthritis synovial fibroblasts encoding a polypeptide having an inhibitory effect on Akt.

17. The vector of claim 16 wherein said polypeptide is selected from the group consisting of: wortmannin, anti-Akt and dominant negative Akt.

18. Use of an Akt inhibitor or a fragment thereof for regulation of rheumatoid arthritis synovial fibroblast apoptosis.

19. A process for assaying rheumatoid arthritis drug candidate apoptosis comprising the steps of:
exposing a culture of rheumatoid arthritis synovial fibroblast to a drug candidate;
monitoring apoptosis of said culture in the presence of said drug candidate; and
comparing apoptosis of said culture to apoptosis induced in a duplicate culture by a control Akt inhibitor.

20. The process of claim 19 wherein said control Akt inhibitor is selected from the group consisting of: wortmannin, celecoxib, SB203580, CGP57148B, and LY294002.

21. Use of an Akt inhibitor for the preparation of a composition for the treatment of rheumatoid arthritis.

22. The use of an Akt inhibitor in the manufacture of a medicament for the treatment of rheumatoid arthritis.

23. A composition as claimed in claim 10 as a rheumatoid arthritis synovial fibroblast apoptosis agent.

24. A process according to claim 1 substantially as described herein with reference to and/or as illustrated in the accompanying drawings.

25. The process of claim 1 wherein said Akt inhibitor is selected from the group consisting of: a cyclooxygenase-2 inhibitor, a pyridinyl imidazole, a tyrosine kinase inhibitor and a PI-3 kinase inhibitor.

26. The process of claim 25 wherein said PI-3 kinase inhibitor is LY294002.

27. The process of claim 25 wherein said pyridinyl imidazole is SB203580.

28. The process of claim 25 wherein said tyrosine kinase inhibitor is

29. The process of claim 25 wherein said cyclooxygenase-2 inhibitor inhibitor is celecoxib.

30. The process of claim 1 wherein said Akt inhibitor is a phosphatase.

31. The process of claim 40 wherein said phosphatase is selected from the group consisting PTEN, a fragment thereof, and a polymorph thereof.
